(19) United States
(12) Patent Application Publication
(10) Pub. No.: US 2026/0018294 A1
(43) Pub. Date: Jan. 15, 2026

(54) Universal tinnitus management system (UTMS)

(71) Applicant: Aureliym GmbH, Bad Neuenahr-Ahrweiler (DE)

(72) Inventors: Christian Hauptmann, Bad Neuenahr-Ahrweiler (DE); Markus Haller, Bad Neuenahr-Ahrweiler (DE); Alexander Wegener, Bad Neuenahr-Ahrweiler (DE)

(21) Appl. No.: 19/336,135

(22) Filed: Sep. 22, 2025

Related U.S. Application Data
(63) Continuation-in-part of application No. 17/635,788, filed on Feb. 16, 2022.

(30) Foreign Application Priority Data
Aug. 16, 2019 (DE) 10 2019 005 768.6

(51) Int. Cl. G16H 50/20; G16H 10/20; G16H 10/60; G16H 20/70; G16H 40/20; G16H 40/67; G16H 80/00

(52) U.S. Cl. CPC G16H 50/20; G16H 10/20; G16H 10/60; G16H 20/70; G16H 40/20; G16H 40/67; G16H 80/00

(57) ABSTRACT

A self-configuring, closed-loop universal tinnitus management system (UTMS) is disclosed, comprising a mobile-implemented patient component, a server, an automated system configuration component, and optionally, a control component for clinician access. The system collects tinnitus-related data through interactive diagnostic modules, categorizes the patient using a multi-stage algorithm, and assigns a corresponding tinnitus-therapy profile. The system automatically configures therapy profile, enables periodic re-categorization, and optionally allows patient-specific adjustments within permitted boundaries.

### RELATED APPLICATIONS

The present application is a continuation-in-part of U.S. patent application Ser. No. 17/635,788, filed Feb. 16, 2022, which is a U.S. National Stage Application under 35 U.S.C. § 371 of International Application No. PCT/EP2020/063433, filed May 14, 2020, which claims priority to DE Patent Application No. 10 2019 005 768, filed Aug. 16, 2019, each of which is hereby incorporated by reference in its entirety.

### TECHNICAL FIELD

The present invention relates to digital healthcare systems and, more particularly, to an autonomous, closed-loop system and method for managing therapy in patients with subjective tinnitus through algorithmic categorization and therapy configuration.

### BACKGROUND INFORMATION

Subjective tinnitus—perception of sound in the absence of external stimuli—affects millions worldwide. Known treatment strategies include cognitive behavioral therapy (CBT), sound therapy (e.g., pink noise, masking, neuromodulation), and patient-specific counseling. However, therapy selection and deployment are largely clinician-dependent and non-adaptive over time. Current mobile applications are typically passive, non-interactive, or require manual therapy configuration.

There remains a need for a system that dynamically personalizes, deploys, and reconfigures tinnitus therapies based on evolving patient-specific data in an autonomous, scalable manner.

### SUMMARY

Described herein is a self-configuring, closed-loop universal tinnitus management system (UTMS) comprising the following components: A patient component is software-implemented on a user-operated mobile device and configured to authenticate the patient to the system; guide the patient through a plurality of software-implemented inter-active diagnostic modules, each implemented as software routines, for acquiring tinnitus-related data of the patient via patient input; transmit the tinnitus-related data to a server. A server is configured to receive and store the data transmitted by the patient component. An automated system configuration component is software-implemented on the mobile device and/or the server and configured to enforce completion of the plurality of interactive diagnostic modules by disabling access to therapeutic modules in the patient component and enabling access to diagnostic modules; process the tinnitus-related data using a predefined multi-stage categorization algorithm, for automatically assigning the patient to one of a plurality of predefined patient categories, each patient category corresponding to a distinct tinnitus-therapy profile involving one or more software-implemented interactive therapeutic modules; enable access to the therapeutic modules in the patient component after patient categorization; automatically configure the patient component, including programming therapy signal generation parameters, based on the assigned patient category, to enable selective access to therapeutic modules involved in the distinct tinnitus-therapy profile corresponding to the assigned patient category; automatically configure the patient component based on the current state of use, to enable access to either diagnostic or therapeutic modules; and automatically enforce scheduled periodic re-categorization, comprising: disabling access to therapeutic modules and enabling access to diagnostic modules in the patient component; acquiring updated tinnitus-related data; reprocessing the updated data using the pre-defined multi-stage categorization algorithm to re-assign a patient category; and reprogramming the therapy signal generation parameters in the patient component based on the re-assigned patient category.

The system guides the patient through interactive diagnostic modules to collect tinnitus-related data, automatically categorizes the patient into a therapy profile, enables therapy module access accordingly, and periodically re-categorizes based on updated data.

In one embodiment, the automated system configuration component is further configured to restrict available modules by disabling access to therapeutic modules in the patient component determined to be contraindicated based on the assigned patient category, and enabling access to therapeutic modules determined to be indicated based on the assigned patient category.

In a further embodiment, the one or more therapeutic modules comprise at least one module selected from the group consisting of: an in-app sound therapy module, a cognitive behavioral therapy (CBT) module, and a masking or neuromodulation module.

In a further embodiment, release of the tinnitus-therapy profile is enabled only upon verification of a valid prescription and a commercial subscription.

In a further embodiment, the system is configured to guide the patient through the diagnostic and/or therapeutic modules autonomously, without human intervention.

In a further embodiment, the predefined multi-stage categorization algorithm uses one or more of a Tinnitus Handicap Inventory (THI) score, a response to psychiatric treatment questions in a Tinnitus Sample Case History Questionnaire (TSCHQ), dependent scores in GAD-7 or PHQ-9 questionnaires, and tonal versus atonal sound evaluation as input variable.

In a further embodiment, the predefined multi-stage categorization algorithm assigns the patient to a first patient category if the tinnitus-related data includes a THI score above a predefined threshold and/or a YES response to psychiatric treatment questions in the TSCHQ, and the scores in GAD-7 or PHQ-9 questionnaires exceed a respective predefined threshold.

In a further embodiment, the one or more therapeutic modules comprise at least one module selected from the group consisting of an internet-based cognitive behavioral therapy (iCBT) module, a pink noise module, a masking therapy module, and a neuromodulation therapy module, each implemented as a software routine and tailored according to category-specific logic.

In a further embodiment, each tinnitus-therapy profile corresponding to one of the patient categories is distinguishable from the therapy profiles associated with other categories in at least one of: the iCBT module, the pink noise module, the masking therapy module, and the neuromodulation therapy mod-ule.

In a further embodiment, the system further comprises a control component software-implemented on a terminal, configured to authenticate a health care professional (HCP) or an ear, nose and throat doctor (ENT), and to enable the HCP or ENT to view and modify therapy signal generation parameters of the assigned therapy profile stored in the patient component.

In a further embodiment, the patient component is further configured to allow the patient, after categorization, to adjust therapy signal generation parameters of the one or more therapeutic modules enabled in the patient component based on the assigned patient category, including (i) for a pink noise module: volume and balance; (ii) for a masking therapy module: volume per frequency band, overall volume, and balance; (iii) for a neuromodulation module: pitch frequency, volume, equal loudness, and balance. The patient-selected therapy signal generation parameters are stored in the patient component and applied during execution of the respective therapeutic modules until a subsequent re-categorization or a manual override command is received from the control component.

A method for personalized tinnitus therapy management using a self-configuring, closed-loop universal tinnitus management system (UTMS) constitutes another aspect described herein. The method comprises the following steps: acquiring tinnitus-related data of a patient from a patient component via patient input; assigning the patient to one of multiple tinnitus-related patient categories based on the acquired patient data, using a predefined multi-stage categorization algorithm; configuring the patient component based on the assigned patient cate-gory, including programming therapy signal generation parameters associated with a tinnitus-therapy profile corresponding to the assigned patient category; enabling selective access to therapeutic modules involved in the tinnitus-therapy profile corresponding to the assigned patient category, optionally contingent on verification of a prescription and subscription; periodically re-categorizing the patient by enforced re-acquisition of tinnitus-related data of the patient via user input in the patient component using software-implemented interactive diagnostic modules, implemented as software routines, and updating the patient category using the predefined multi-stage categorization algorithm; reprogramming the therapy signal generation parameters based on the updated patient category; and storing categorization and re-categorization results and therapy signal generation parameter adjustments in a database on a server.

Yet another aspect pertains to a computer-implemented method for configuring a tinnitus therapy device, comprising: receiving tinnitus-related data of a patient via user input in response to questionnaires and hearing performance tests displayed or played on a patient component; processing the patient-specific data to assign the patient to one of a plurality of patient categories based on predefined score thresholds and tonal/atonal classification; configuring the patient component by programming therapy signal generation parameters associated with a distinct tinnitus-therapy profile corresponding to the assigned category and measured hearing performance; enabling access to at least one therapy module involved in the distinct tinnitus-therapy profile, based on the assigned category, wherein the at least one therapy module is selected from the group of: cognitive behavioral therapy, pink noise, masking, and neuromodulation; after a predefined interval, re-acquiring updated patient-specific data including a Tinnitus Handicap Inventory (THI) score; determining a trend based on a change in the THI score; and based on the trend, automatically continuing the therapy, or reconfiguring the patient component by reprogramming the therapy signal generation parameters in the patient component, the reprogrammed therapy signal generation parameters being associated with a distinct tinnitus-therapy profile corresponding to a re-assigned category. The therapy signal generation parameters are programmed and reprogrammed in the patient component without human intervention unless a manual override command is received from a control component.

Finally, it is noted that each feature described in relation to a specific aspect or embodiment is meant to be combinable with other aspects or embodiments, unless the combination is technically meaningless or impossible.

### BRIEF DESCRIPTION OF THE DRAWINGS

The inventive concept, non-limiting embodiments, and further advantages of the inventive concept will now be described with reference to the drawings in which:

FIG. 1 illustrates a block diagram showing the main system components, including the patient component, server, automated system configuration component, and control terminal.

FIG. 2 is a flowchart illustrating the diagnostic-to-therapy deployment logic, including categorization, therapy signal configuration, and periodic re-categorization.

FIG. 3 shows a diagram of patient adjustment interface showing configurable therapy signal parameters for pink noise, masking, and neuromodulation modules.

FIG. 4 illustrates a logic flow of the multi-stage categorization algorithm showing threshold comparisons and dependent evaluation of inputs (e.g., THI, TSCHQ, GAD-7, tonal classification).

### DETAILED DESCRIPTION

The description of the invention to be provided herein is given purely by way of example and is not to be taken in any way as limiting the scope or extent of the invention. The invention will be described with reference to the drawings.

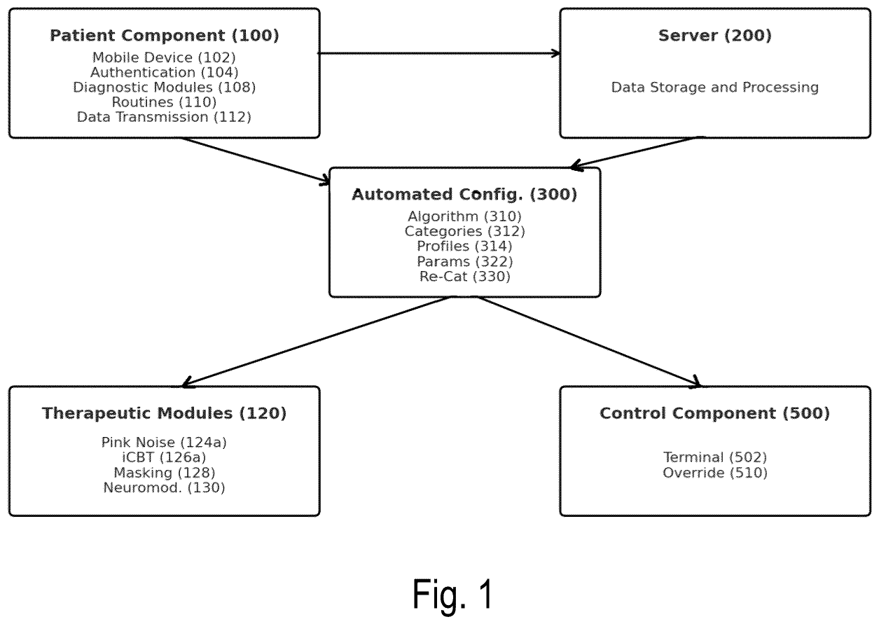

Referring now to FIG. 1, there is illustrated a self-configuring, closed-loop universal tinnitus management system (UTMS) (10) for treatment of subjective tinnitus.

The system (10) comprises a patient component (100) software-implemented on a user-operated mobile device (102), such as a smartphone or tablet. The patient component (100) is configured to: authenticate the patient (104) to the system, e.g., by password, biometric identifier, or secure token; guide the patient (106) through a plurality of software-implemented interactive diagnostic modules (108), each diagnostic module implemented as a discrete software routine (110), for acquiring tinnitus-related data of the patient via patient input; and transmit the tinnitus-related data (112) to a remote or local server (200).

The system further comprises a server (200) configured to receive and store the tinnitus-related data (112) transmitted by the patient component (100) The server (200) provides secure storage and may host additional processing functions.

The system (10) also comprises an automated system configuration component (300), software-implemented either on the patient device (102), the server (200), or distributed across both.

The automated system configuration component (300) is configured to: enforce completion of the plurality of diagnostic modules (108) by disabling access to therapeutic modules (120) in the patient component (100) and enabling access only to diagnostic modules (108) until completion is verified; process the tinnitus-related data (112) using a predefined multi-stage categorization algorithm (310) to automatically assign the patient to one of a plurality of predefined patient categories (312), each patient category corresponding to a distinct tinnitus-therapy profile (314) involving one or more software-implemented interactive therapeutic modules (120); enable access to the therapeutic modules (120) in the patient component (100) after categorization has been completed; automatically configure the patient component (100), including programming therapy signal generation parameters (322), based on the assigned patient category (312), so as to enable selective access to therapeutic modules (120) corresponding to the distinct tinnitus-therapy profile (314) of that patient category; automatically configure the patient component (100) based on the current state of use (324), thereby enabling either diagnostic mode (modules 108) or therapeutic mode (modules 120); and automatically enforce scheduled periodic re-categorization (330). Scheduled periodic re-categorization (330) comprises disabling access to therapeutic modules (120) and enabling access to diagnostic modules (108) in the patient component (100); acquiring updated tinnitus-related data (332); reprocessing the updated data using the multi-stage categorization algorithm (310) to re-assign the patient category (312); and reprogramming the therapy signal generation parameters (322) in the patient component (100) based on the re-assigned patient category.

Advantageously, the system (10) ensures that therapy is always conditioned on up-to-date diagnostic intake, patient categorization, and system-enforced configuration, while maintaining a closed feedback loop that cycles through diagnosis, categorization, therapy deployment, and re-categorization. Thereby, the features described herein enable scalable, personalized tinnitus management with minimal clinician intervention, supporting continuous adaptation of therapy.

### Automated Module Restriction Logic (344)

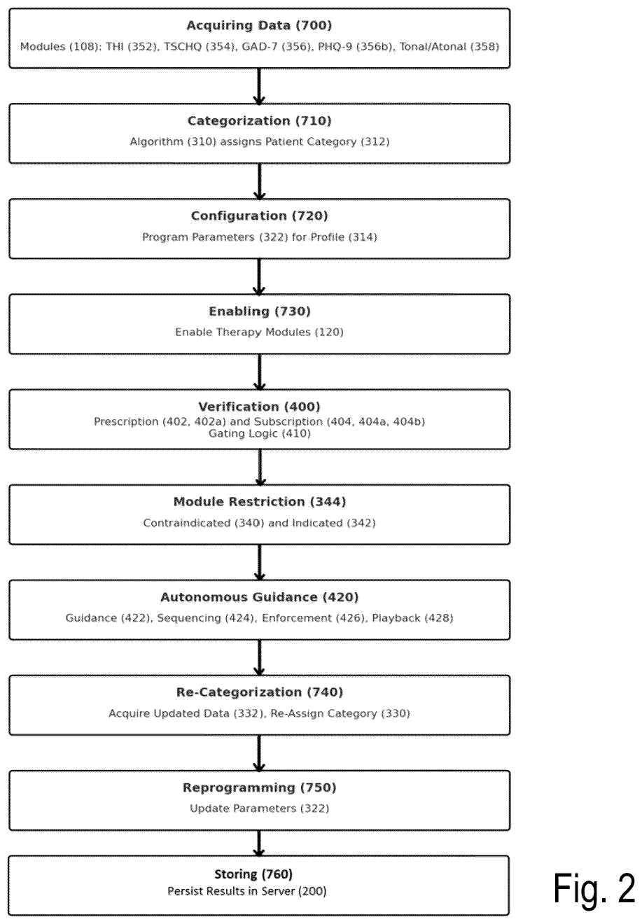

As illustrated in FIG. 2, the automated system configuration component (300) is further configured to restrict availability of the therapeutic modules (120) within the patient component (100).

In an exemplary execution of such module restriction logic (344), the system determines which therapy options are indicated (342) and which are contraindicated (340) based on the output of the multi-stage categorization algorithm (310) and the assigned patient category (312). If a therapy module is determined to be contraindicated (340) for the assigned patient category (312), access to that module can be disabled in the patient component (100). For example, neuromodulation may be contraindicated (340) in patients with certain psychiatric comorbidities. Conversely, therapy modules determined to be indicated (342) can be enabled in the patient component (100). Examples include enabling internet-based CBT (126) for patients with high THI and YES TSCHQ psychiatric responses, enabling pink noise therapy (124a) or masking therapy (128) for tonal tinnitus categories, or enabling neuromodulation therapy (130) where evidence supports efficacy for the assigned patient category.

This module restriction logic (344) ensures that the patient only has access to therapies that are appropriate for their specific categorization, thereby reducing risk of misuse and ensuring compliance with medically validated therapy profiles.

The module restriction logic (344) can be maintained dynamically: During initial configuration, modules may be enabled/disabled according to category-specific logic. During re-categorization cycles, the module restriction logic (344) may be reapplied, ensuring that if the patient's category changes, the enabled/disabled set of therapeutic modules is automatically updated.

### Therapeutic Modules (120)

Referring to FIG. 1 and FIG. 2, the system (10) provides access to a plurality of therapeutic modules (120) implemented as software routines within the patient component (100). These modules are configured to deliver distinct therapeutic modalities and may be selectively enabled or disabled based on patient category (312).

According to an embodiment, the therapeutic modules (120) may include at least one of the following:

In-App Sound Therapy Module (124): This module provides sound-based interventions such as pink noise (124a) or broadband masking noise (124b). It is configured to output audio signals through the patient's mobile device (102) speakers or connected headphones. Therapy signal generation parameters (322) such as volume, balance, and frequency distribution are applied to the audio output.

Internet-Based Cognitive Behavioral Therapy (iCBT) Module (126), implemented as a software routine, delivers structured CBT interventions adapted for tinnitus patients. It provides educational content, coping strategies, relaxation exercises, and interactive cognitive restructuring, and can be delivered via in-app multimedia (text, audio, video) via interactive exercises, quizzes, or video/audio instructions.

Masking Module (128): The masking module (128) generates frequency-specific masking noise to reduce perception of tinnitus tones. Therapy signal generation parameters (322) include volume per band (128a) and overall balance (128b), which may be pre-set by the system or adjusted by the patient under system control.

Neuromodulation Module (130): It delivers auditory neuromodulation signals designed to reduce aberrant neuronal activity linked to tinnitus perception. Configurable therapy signal generation parameters (322) include pitch frequency (130a), signal amplitude (130b), and equal loudness adjustment (130c).

Each of the therapeutic modules (120) is associated with category-specific logic (314), as defined herein. For example, a patient categorized as tonal tinnitus may receive masking noise (128). A patient with psychological comorbidity may receive iCBT (126). A patient with severe high-THI scores may be assigned neuromodulation (130) in combination with sound therapy (124).

### Prescription and Subscription Verification (400)

As illustrated in FIG. 2, the system (10) may include a verification module (400) integrated within the automated system configuration component (300). The verification module (400) ensures that access to a tinnitus-therapy profile (314) is conditional on compliance with external authorization requirements.

The verification module (400) comprises prescription verification (402, 402a) and subscription verification (404, 404a, 404b).

Prescription verification (402) may be implemented by linking the system to an electronic prescription database or clinician input via the control component (500). A valid prescription (402a) must be confirmed before therapy modules (120) are enabled. This mechanism is particularly useful in countries where neuromodulation (130) or advanced iCBT modules (126) require medical authorization.

The system may further require confirmation of an active commercial subscription (404a). Subscription verification (404) may occur through a secure server (200) linked to a billing database (404b). Without a valid subscription, therapeutic modules (120) remain locked, even if categorization (312) has been completed.

The verification module (400) may implement gating logic (410): If both prescription verification (402) and subscription verification (404) are satisfied, the assigned therapy profile (314) is released to the patient component (100). If either verification step fails, therapy modules (120) remain disabled, even after categorization by the multi-stage algorithm (310).

During initial setup, therapy signal generation parameters (322) are programmed but not activated until verification (400) is complete. During re-categorization cycles, verification status may be re-checked before new therapy modules are enabled.

### Autonomous Guidance Logic (420)

As illustrated in FIG. 2, the automated system configuration component (300) may further comprise autonomous guidance logic (420). This logic ensures that the patient component (100) can guide the patient through diagnostic modules (108) and therapeutic modules (120) without manual input or supervision by a clinician.

Guidance may include step-by-step instructions (422), automated module sequencing (424), and automated enforcement (426). Step-by-step instructions (422) include prompts, questionnaires, and visual/audio cues that guide the patient through diagnostics (e.g., THI, TSCHQ, GAD-7). Automated module sequencing (424) means that the system dynamically unlocks the next required diagnostic module (108) or therapy module (120) according to system state. Automated enforcement (426) means that diagnostic modules (108) must be completed before therapy modules (120) can be accessed, ensuring compliance with categorization requirements.

### Therapeutic Module Autonomy

Once a patient category (312) is assigned, the therapy profile (314) is automatically deployed. Therapy modules (124-130) provide interactive content or sound playback autonomously (428). No clinician intervention is required to adjust module access, initiate sound therapy, or deliver CBT exercises, unless a manual override is initiated through control component (500).

During initial onboarding the patient is autonomously guided through authentication (104), diagnostics (108), categorization (310), and initial therapy profile (314) deployment. During re-categorization cycles (330) the patient is autonomously prompted to complete reassessment, with therapy modules (120) disabled until re-categorization is complete. Thereby, the system provides autonomous patient guidance through diagnostic and therapy modules, enforcing the correct sequence and ensuring closed-loop operation, without human intervention except where expressly allowed through control override.

### Multi-Stage Categorization Algorithm (310)

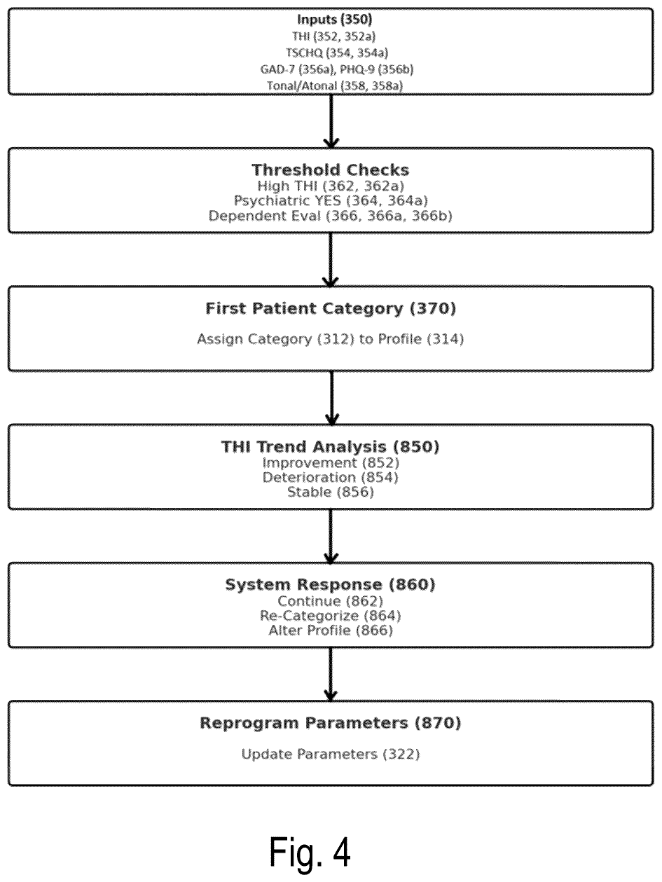

As illustrated in FIG. 4, the multi-stage categorization algorithm (310) operates within the automated system configuration component (300). The algorithm processes tinnitus-related patient data as input variables (350) derived from diagnostic modules (108) in the patient component (100).

The following diagnostic data may serve as inputs to the categorization algorithm (310):

THI Score (352), derived from patient responses to the Tinnitus Handicap Inventory (THI) questionnaire (352a). It provides a numeric severity measure that reflects the degree of functional and emotional handicap caused by tinnitus.

TSCHQ Psychiatric Responses (354), obtained from the Tinnitus Sample Case History Questionnaire (TSCHQ) (354a). It includes binary or scaled responses to psychiatric treatment history or comorbidities. Positive psychiatric responses may trigger dependent logic in subsequent stages (described further below).

GAD-7 and PHQ-9 Dependent Scores (356): When psychiatric flags are raised in the TSCHQ, the system administers additional questionnaires, the Generalized Anxiety Disorder score—GAD-7 (356a)—and the Patient Health Questionnaire for depression—PHQ-9 (356b). These dependent scores serve as additional thresholds for classification.

Tonal vs. Atonal Evaluation (358), collected through an interactive tonal classification module (358a) in the patient component (100). It determines whether tinnitus is perceived as tonal (continuous pure tone) or atonal (broadband, hissing, or noise-like). This evaluation influences therapy selection, e.g., masking vs. neuromodulation.

The categorization algorithm (310) may accept one or more of these input variables (350) and may apply decision-tree logic to assign the patient to a category (312). The algorithm may support weighted or sequential processing, ensuring that multiple input types (e.g., THI+tonal classification) can be combined for refined categorization.

### Threshold-Based Categorization (360)

As illustrated in FIG. 4, the multi-stage categorization algorithm (310) may apply threshold-based categorization logic (360) to assign patients into categories (312).

According to a specific example, the algorithm assigns the patient to a first patient category (370) when the following conditions are met:

High THI Score (362): If the patient's THI score (352) exceeds a predefined severity threshold (362a), the algorithm triggers assignment to the first patient category (370). For example, a THI>58 may represent severe handicap.

Positive TSCHQ Psychiatric Response (364): If the patient responds YES (364a) to psychiatric treatment questions in the TSCHQ (354), this flag activates dependent evaluation. An example is where the patient confirms history of psychiatric intervention.

Dependent GAD-7/PHQ-9 Evaluation (366): If TSCHQ responses (354) indicate psychiatric factors, the system then evaluates the GAD-7 (356a) and PHQ-9 (356b) scores. Assignment to the first patient category (370) requires that one or both exceed a respective predefined threshold (366a, 366b). A representative example result could be GAD-7>10 or PHQ-9>15.

If one or more of the above criteria are satisfied, the algorithm assigns the patient to the first patient category (370). This category corresponds to a therapy profile (314) emphasizing psychological intervention and potentially combined with sound therapy (124). For instance, high-THI patients with psychiatric comorbidities may receive iCBT (126) as a primary module, combined with masking (128).

During re-categorization, the same thresholds (362, 364, 366) may be reapplied. Patients who show improvement (e.g., THI dropping below threshold 362a) may be reassigned out of the first patient category (370) into a less intensive therapy profile.

### Configurable Therapy Signal Generation Parameters (322)

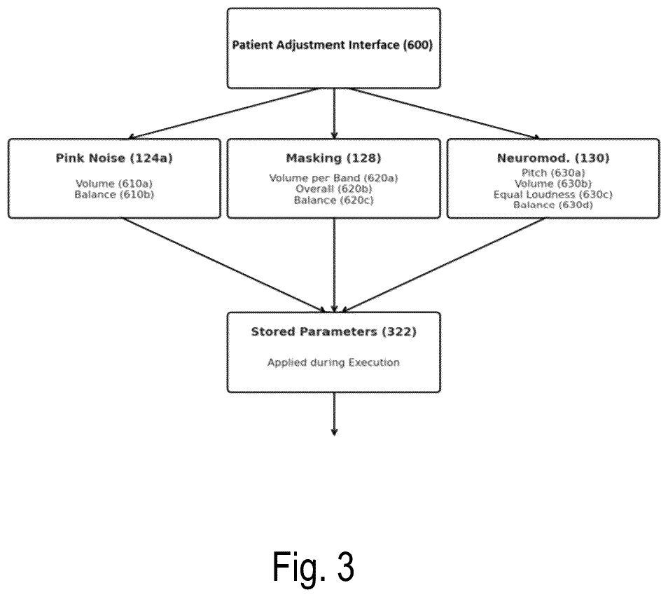

As illustrated in FIG. 1 and FIG. 3, the therapeutic modules (120) are software-implemented routines within the patient component (100). Each therapeutic module corresponds to a specific therapy modality and is automatically configured according to the patient's assigned therapy profile (314). In addition, the system may allow patient adjustments of therapy signal generation parameters (322).

Configurable therapy signal generation parameters (322) of the pink noise module (124a) may include overall volume, left/right channel balance, and duration of playback.

Configurable therapy signal generation parameters (322) of the masking therapy module (128) may include frequency-band-specific volume controls (128a) and overall balance (128b).

Configurable signal generation parameters (322) of the neuromodulation module (130) may include pitch frequency (130a), amplitude (130b), and equal loudness balancing (130c).

### Category-Specific Tailoring (314)

Each therapeutic module (124, 126, 128, 130) is adapted based on the assigned patient category (312) determined by the multi-stage categorization algorithm (310). Exemplary category-specific tailoring can be implemented as follows: High-THI/psychiatric comorbidity: iCBT (126) prioritized, with optional pink noise (124a). Tonal tinnitus: masking therapy (128) emphasized. Atonal tinnitus: pink noise (124a) or broadband sound therapy used. Severe tinnitus with neurological markers: neuromodulation (130) deployed.

### Tinnitus-Therapy Profiles (314)

As illustrated in FIG. 2 and FIG. 3, the system (10) comprises a plurality of tinnitus-therapy profiles (314), each corresponding to a patient category (312) assigned by the categorization algorithm (310). Each therapy profile (314) is distinguished from all others by at least one differentiating feature (380) in the configuration of the therapeutic modules (120).

Examples of profile differentiation (380) include: iCBT Module (126): Profile A: iCBT unlocked, full CBT program including mood tracking. Profile B: iCBT locked, or only limited CBT routines enabled. Pink Noise Module (124a): Profile C: Pink noise enabled with balanced full-spectrum output. Profile D: Pink noise output adjusted to emphasize low-frequency masking. Masking Therapy Module (128): Profile E: Narrowband masking noise centered on tonal tinnitus frequency. Profile F: Broadband masking covering multiple frequency bands. Neuromodulation Module (130): Profile G: Neuromodulation enabled with default pitch and amplitude. Profile H: Neuromodulation disabled, replaced with alternative sound therapy.

By varying which modules (120) are enabled and how their therapy signal generation parameters (322) are configured, each therapy profile (314) is unique. This ensures that two patients in different categories (312) will not receive identical therapy module sets or parameter settings.

During re-categorization (330), if the patient is reassigned to a different category (312), the therapy profile (314) changes accordingly. The differences across profiles guarantee that re-assignment has a concrete effect on therapy delivery.

### Control Component (500)

As illustrated in FIG. 1, the system (10) may further comprise a control component (500), software-implemented on a terminal (502). The terminal (502) may be a clinician's computer, tablet, or secure workstation connected via a network to the server (200) and patient component (100). The control component (500) provides an interface for authorized healthcare professionals.

The control component (500) may include an authentication module (504) to verify the identity of a healthcare professional (HCP) or an ear, nose and throat doctor (ENT). Authentication may use login credentials, two-factor authentication, or secure digital certificates. In some examples, only authenticated users may gain access to patient-specific therapy data.

Once authenticated, the HCP/ENT is permitted to view therapy data (506) including one or more of patient category (312), current tinnitus-therapy profile (314), active therapeutic modules (120), configured therapy signal generation parameters (322), historical therapy adjustments and re-categorization results.

Modification function (508) of the control component (500) also enables the HCP/ENT to modify therapy signal generation parameters (322) of the assigned therapy profile (314). Modifications may include one or more of adjusting pink noise balance (124a), disabling or enabling masking therapy (128), changing neuromodulation frequency (130a), overriding patient adjustments. Modified parameters are stored in the patient component (100) and may take effect immediately or at the next therapy cycle.

The manual override command (510) from the control component (500) supersedes automated categorization (310) or patient adjustments. During periodic re-categorization (330), clinician modifications may remain valid unless contradicted by updated medical guidance, or may be reset according to the updated therapy profile (314).

### Patient Adjustment Interface (600)

As illustrated in FIG. 3, the patient component (100) may further include a patient adjustment interface (600). The interface (600) becomes available only after categorization (310) has assigned the patient to a category (312) and enabled the corresponding therapy modules (120). The interface provides interactive controls (sliders, buttons, drop-downs) for adjusting permitted therapy signal generation parameters (322). Pink noise adjustments (610): For the pink noise module (124a), the patient may adjust volume (610a), channel balance (610b) between left and right ears. Masking noise adjustments (620). For the masking module (128), the patient may adjust volume per frequency band (620a), overall volume (620b), channel balance (620c). Neuromodulation adjustments (630): For the neuromodulation module (130), the patient may adjust, as therapy signal parameters (322), pitch frequency (630a), signal amplitude/volume (630b), equal loudness settings (630c), balance between ears (630d).

The patient-selected therapy signal generation parameters (322) are stored locally in the patient component (100). They are applied during execution of the respective therapeutic module (120) until a subsequent re-categorization (330) occurs, which resets parameters (322) according to the updated therapy profile (314), or a manual override command (510) is received from the control component (500).

Allowing the patient to make adjustments, patient autonomy is provided within system-defined limits, ensuring personalization of therapy while maintaining medical safety. If re-categorization indicates contraindications, adjustments made in interface (600) may be automatically reset or restricted.

### Method for Personalized Tinnitus Therapy Management

Another aspect of the features described herein is a method for personalized tinnitus therapy management using a self-configuring, closed-loop universal tinnitus management system (UTMS) as defined herein. The method comprises steps of acquiring tinnitus-related data, assigning a patient category, configuring patient component, enabling selective access, periodically re-categorizing, reprogramming parameters (322), and storing results. These steps are further described below.

Acquiring tinnitus-related data (700): Referring to FIG. 2, the method begins with data acquisition (700). The patient component (100) presents interactive diagnostic modules (108). Patient responses are collected from THI (352), TSCHQ (354), GAD-7/PHQ-9 (356), and tonal/atonal evaluation (358). Data are transmitted to the server (200) for storage and further processing.

Assigning patient category (710): The multi-stage categorization algorithm (310) processes the acquired input variables (350). Based on thresholds (362, 364, 366), the system assigns the patient to one of the predefined patient categories (312). Each category maps to a distinct therapy profile (314).

Configuring patient component (720): The automated system configuration component (300) automatically configures the patient component (100) according to the assigned profile (314). This includes programming therapy signal generation parameters (322) relevant for the selected modules (124, 126, 128, 130).

Enabling selective access (730): Once configured, the system enables selective access (730) to therapeutic modules (120) corresponding to the patient's assigned profile (314). Access remains gated by verification module (400), ensuring prescription (402) and subscription (404) requirements are met.

Periodic re-categorization (740): At scheduled intervals, the system enforces periodic re-categorization (740). Therapy modules (120) are temporarily disabled, while diagnostic modules (108) are re-enabled. The patient re-completes the THI, TSCHQ, and other inputs (350).

Reprogramming parameters (750): The categorization algorithm (310) processes the updated tinnitus-related data (332). Therapy signal generation parameters (322) are reprogrammed according to the new or confirmed patient category (312). Adjustments propagate directly to the patient component (100) for therapy playback.

[symbol]

results (760): Categorization outcomes, re-categorization outcomes, and therapy signal adjustments are stored in the server database (200). Storage provides traceability for both autonomous operations and HCP review (via control component 500).

The method ensures continuous personalization by cycling through steps

700→710→720→730→740→750→760,

implementing a closed-loop therapy management system.

At all times, human intervention is optional, not required (except for control override).

Another related aspect of the features described herein is a computer-implemented method for configuring a tinnitus therapy device, comprising the following steps.

Acquiring patient data (800): Referring to FIG. 2, the method begins with acquiring patient data (800). To this end, the patient component (100) may present questionnaires and hearing performance tests (802). Responses are captured as tinnitus-related data (350) and may include THI (352), TSCHQ (354), GAD-7/PHQ-9 (356), and tonal/atonal classification (358).

Processing and categorization (810): The multi-stage categorization algorithm (310) processes input variables (350). The patient is assigned to one of a plurality of patient categories (312) using predefined thresholds (362, 364, 366). Tonal vs. atonal classification (358) may determine whether masking (128) or pink noise (124a) is favored.

Configuring patient component (820): The automated system configuration component (300) configures the patient component (100) by programming therapy signal generation parameters (322). Parameters correspond to the assigned therapy profile (314) and measured hearing performance (802).

Enabling therapy modules (830): The assigned therapy profile (314) enables access to one or more therapy modules (120) selected from iCBT (126), pink noise (124a), masking (128), or neuromodulation (130). Access may be restricted by the verification module (400) until prescription (402) and subscription (404) are validated.

Re-Acquiring data after interval (840): After a predefined interval (e.g., weeks), the patient is prompted to complete updated diagnostics. Re-acquired data (332) includes a new THI score (352) and may include updated TSCHQ (354) or tonal classification (358).

Determining THI trend (850): A trend analysis module (850) compares the updated THI score to previous scores stored in the server (200). The system determines whether the THI shows improvement (852) (downward trend), deterioration (854) (upward trend), or no change (856) (stable values).

Automated response (860): Based on THI trend (850), the system may execute (i) continuation of the current therapy (862) (therapy modules (120) persist unchanged), if trend shows improvement; (ii) re-categorization (864), if trend shows deterioration; (iii) alteration of therapy profile (866) by modifying therapy signal generation parameters (322) (e.g., adjust masking balance, neuromodulation pitch), if trend shows no significant change.

Programming/reprogramming parameters (870): Therapy parameters (322) are programmed and reprogrammed (870) in the patient component (100) automatically. This occurs without human intervention, ensuring continuity of therapy. If deemed indicative, a manual override command (510) from the control component (500) can supersede system decisions.

Steps (800→870) repeat in cycles, forming a closed-loop therapy process that adapts therapy dynamically to patient progress. The loop guarantees therapy profiles evolve according to both categorization logic (310) and longitudinal THI trend (850).

What is claimed is:

1. A self-configuring, closed-loop universal tinnitus management system (UTMS) for treatment of subjective tinnitus, the system comprising:
a) a patient component software-implemented on a user-operated mobile device and configured to:
a1) authenticate the patient to the system;
a2) guide the patient through a plurality of software-implemented interactive diagnostic modules, each implemented as software routines, for acquiring tinnitus-related data of the patient via patient input;
a3) transmit the tinnitus-related data to a server;
b) a server configured to receive and store the data transmitted by the patient component;
c) an automated system configuration component software-implemented on the mobile device and/or the server, configured to:
c1) enforce completion of the plurality of interactive diagnostic modules by disabling access to therapeutic modules in the patient component and enabling access to diagnostic modules;
c2) process the tinnitus-related data using a predefined multi-stage categorization algorithm, for automatically assigning the patient to one of a plurality of predefined patient categories, each patient category corresponding to a distinct tinnitus-therapy profile involving one or more software-implemented interactive therapeutic modules;
c3) enable access to the therapeutic modules in the patient component after patient categorization;
c4) automatically configure the patient component, including programming therapy signal generation parameters, based on the assigned patient category, to enable selective access to therapeutic modules involved in the distinct tinnitus-therapy profile corresponding to the assigned patient category;
c5) automatically configure the patient component based on a current state of use, to enable access to either diagnostic or therapeutic modules; and
c6) automatically enforce scheduled periodic re-categorization, comprising: disabling access to therapeutic modules and enabling access to diagnostic modules in the patient component; acquiring updated tinnitus-related data; reprocessing the updated data using the predefined multi-stage categorization algorithm to re-assign a patient category; and reprogramming the therapy signal generation parameters in the patient component based on the re-assigned patient category.

2. The system of claim 1, wherein the automated system configuration component is further configured to restrict available modules by disabling access to therapeutic modules in the patient component determined to be contraindicated based on the assigned patient category, and enabling access to therapeutic modules determined to be indicated based on the assigned patient category.

3. The system of claim 1, wherein the one or more therapeutic modules comprise at least one module selected from the group consisting of: an in-app sound therapy module, a cognitive behavioral therapy (CBT) module, and a masking or neuromodulation module.

4. The system of claim 1, wherein release of the tinnitus-therapy profile is enabled only upon verification of a valid prescription and a commercial subscription.

5. The system of claim 1, wherein the system is configured to guide the patient through the diagnostic and/or therapeutic modules autonomously, without human intervention.

6. The system of claim 1, wherein the predefined multi-stage categorization algorithm uses one or more of a Tinnitus Handicap Inventory (THI) score, a response to psychiatric treatment questions in a Tinnitus Sample Case History Questionnaire (TSCHQ), dependent scores in GAD-7 or PHQ-9 questionnaires, and tonal versus atonal sound evaluation as input variable.

7. The system of claim 6, wherein the predefined multi-stage categorization algorithm assigns the patient to a first patient category if the tinnitus-related data includes a THI score above a predefined threshold and/or a YES response to psychiatric treatment questions in the TSCHQ, and the scores in GAD-7 or PHQ-9 questionnaires exceed a respective predefined threshold.

8. The system of claim 1, wherein the one or more therapeutic modules comprise at least one module selected from the group consisting of an internet-based cognitive behavioral therapy (iCBT) module, a pink noise module, a masking therapy module, and a neuromodulation therapy module, each implemented as a software routine and tailored according to category-specific logic.

9. The system of claim 8, wherein each tinnitus-therapy profile corresponding to one of the patient categories is distinguishable from the therapy profiles associated with other categories in at least one of: the iCBT module, the pink noise module, the masking therapy module, and the neuromodulation therapy module.

10. The system of claim 1, further comprising a control component software-implemented on a terminal, configured to authenticate a health care professional (HCP) or an ear, nose and throat doctor (ENT), and to enable the HCP or ENT to view and modify therapy signal generation parameters of the assigned therapy profile stored in the patient component.

11. The system of claim 1, wherein the patient component is further configured to allow the patient, after categorization, to adjust therapy signal generation parameters of the one or more therapeutic modules enabled in the patient component based on the assigned patient category, including:
for a pink noise module: volume and balance;
for a masking therapy module: volume per frequency band, overall volume, and balance;
for a neuromodulation module: pitch frequency, volume, equal loudness, and balance;
wherein the patient-selected therapy signal generation parameters are stored in the patient component and applied during execution of the respective therapeutic modules until a subsequent re-categorization or a manual override command is received from a control component.

12. A method for personalized tinnitus therapy management using a self-configuring, closed-loop universal tinnitus management system (UTMS) as defined in claim 1, the method comprising:
acquiring tinnitus-related data of a patient from a patient component via patient input;
assigning the patient to one of multiple tinnitus-related patient categories based on the acquired patient data, using a predefined multi-stage categorization algorithm;
configuring the patient component based on the assigned patient category, including programming therapy signal generation parameters associated with a tinnitus-therapy profile corresponding to the assigned patient category;
enabling selective access to therapeutic modules involved in the tinnitus-therapy profile corresponding to the assigned patient category, optionally contingent on verification of a prescription and subscription;
periodically re-categorizing the patient by enforced re-acquisition of tinnitus-related data of the patient via user input in the patient component using software-implemented interactive diagnostic modules, implemented as software routines, and updating the patient category using the predefined multi-stage categorization algorithm;
reprogramming the therapy signal generation parameters based on the updated patient category; and
storing categorization and re-categorization results and therapy signal generation parameter adjustments in a database on a server.

13. A computer-implemented method for configuring a tinnitus therapy device, comprising:
receiving tinnitus-related data of a patient via user input in response to questionnaires and hearing performance tests displayed or played on a patient component;
processing the patient-specific data to assign the patient to one of a plurality of patient categories based on predefined score thresholds and tonal/atonal classification;
configuring the patient component by programming therapy signal generation parameters associated with a distinct tinnitus-therapy profile corresponding to the assigned category and measured hearing performance;
enabling access to at least one therapy module involved in the distinct tinnitus-therapy profile, based on the assigned category, wherein the at least one therapy module is selected from the group of: cognitive behavioral therapy, pink noise, masking, and neuromodulation;
after a predefined interval, re-acquiring updated patient-specific data including a Tinnitus Handicap Inventory (THI) score;
determining a trend based on a change in the THI score; and
based on the trend, automatically continuing the therapy, or reconfiguring the patient component by reprogramming the therapy signal generation parameters in the patient component, the reprogrammed therapy signal generation parameters being associated with a distinct tinnitus-therapy profile corresponding to a re-assigned category,
wherein the therapy signal generation parameters are programmed and reprogrammed in the patient component without human intervention unless a manual override command is received from a control component.
